(19) United States
(12) Patent Application Publication
(10) Pub. No.: US 2023/0203595 A1
(43) Pub. Date: Jun. 29, 2023

(54) SURFACE-ENHANCED RAMAN SCATTERING (SERS) BIOSENSOR FOR DIAGNOSING PROSTATE CANCER WITH HIGH SENSITIVITY

(71) Applicant: Korea University Research and Business Foundation, Seoul (KR)

(72) Inventors: Sang Jun SIM, Seoul (KR); Jong Uk LEE, Seoul (KR); Woo Hyun KIM, Namyangju-si (KR)

(73) Assignee: Korea University Research and Business Foundation, Seoul (KR)

(21) Appl. No.: 17/984,444

(22) Filed: Nov. 10, 2022

(30) Foreign Application Priority Data
Nov. 11, 2021 (KR) 10-2021-0154725
Oct. 24, 2022 (KR) 10-2022-0137120

(51) Int. Cl. C12Q 1/6886; G01N 21/65

(52) U.S. Cl. CPC C12Q 1/6886; G01N 21/658; C12Q 2600/178; C12Q 1/6825

(57) ABSTRACT

Disclosed are a surface-enhanced Raman scattering-based biosensor for diagnosing prostate cancer with high sensitivity and a method of detecting a prostate-cancer-derived target biomarker using the same.

### CROSS-REFERENCE TO RELATED APPLICATIONS

This application claims the benefit under 35 USC § 119(a) of Korean Patent Application No. 10-2021-0154725 filed on Nov. 11, 2021 and Korean Patent Application No. 10-2022-0137120 filed on Oct. 24, 2022 in the Korean Intellectual Property Office, the entire disclosures of which are incorporated herein by reference for all purposes.

### BACKGROUND OF THE INVENTION

### Field of the Invention

The present invention relates to a surface-enhanced Raman scattering (SERS) biosensor for diagnosing prostate cancer with high sensitivity and a method of detecting a prostate-cancer-derived target biomarker using the same.

### Description of the Related Art

Prostate cancer (PC) is the second most common malignancy in men worldwide and the fifth leading cause of cancer-related death. Serum prostate-specific antigen (PSA) testing is currently the standard for diagnosis and prognosis of PC. However, many studies have reported that the PSA test for PC lacks specificity and accuracy, resulting in a high false-positive rate of up to 76% (Mazzucchelli et al. 2000; Thompson et al. 2006; Woolf 1995). This high false-positive rate has increased the number of unnecessary biopsies. Moreover, inability to distinguish between indolent and aggressive PCs using PSA as a biomarker increased the likelihood of overdiagnosis and overtreatment. Hence, there is an urgent need to develop an accurate and reliable PC diagnostic platform using a new PC-specific biomarker.

According to recent studies, tumor-derived exosomes are reported to be involved in cancer pathogenesis, including tumor growth, metastasis, and tumor angiogenesis. These nanovesicles are known to induce malignant transformation of target cells by mediating intercellular communication in the tumor microenvironment and delivering cancer-specific molecular markers (including proteins and nucleic acids) from tumor cells to remote normal cells. In addition, it is known that tumor cells secrete exosomes in an amount corresponding to at least 10 times that of normal cells (Pan et al. 2019). Intensive studies on the components present in exosomes, particularly exosomal miRNAs, involved in tumor progression, have followed.

Exosomal miRNAs are small non-coding single-stranded RNAs that regulate gene expression at post-transcriptional stages. Thereamong, urinary exosomal miRNA was investigated as a new biomarker for PC diagnosis with large-scale quantity and stability (Kim et al. 2021). Also, exosomal miRNA-21 (miR-21) among exosomal miRNAs was identified as an oncogene for PC, which is reported to promote PC cell proliferation and invasion by regulating the expression of several tumor-related genes through the p53 network (Gao et al. 2016; Zhan et al. 2018). In addition, exosomal miRNA-10a (miR-10a) in PC was recently found to be oncogenic miRNA (Zimta et al. 2019), which is reported to be released from cancer cells by inducing conversion of normal cells into cancer stem cells (Ngalame et al. 2018). For this reason, many studies have developed miR-10a and miR-21 detection platforms for PC diagnosis.

Conventional methods for detecting miRNA in blood or biofluids include Northern blotting (Varallyay et al. 2008), qRT-PCR (Varkonyi-Gasic et al. 2007; Wang and Yang 2010), microarray (Zhao et al. 2012), and fluorescence. However, these methods are time-consuming and require lengthy protocols and high costs due to use of fluorescent labeling. Moreover, the conventional methods are problematic in that an additional amplification process is required for the diagnosis of prostate cancer in exosomes containing biofluids (ranging from aM to fM) due to low sensitivity or large amounts of samples have to be used (Campuzano et al. 2014; Labib et al. 2013). Therefore, a sensing platform having superior performance for diagnosing prostate cancer with high sensitivity and high selectivity in a wide dynamic detection range through a simple pretreatment process is still urgently needed.

Meanwhile, surface-enhanced Raman scattering (SERS)-based optical biosensors using noble metal substrates or nanoparticles provide specific “fingerprint” spectral profiles for adsorbed individual bioanalytes (protein, DNA, and RNA), and thus have received great attention as promising technology. Signals from metal nanostructures are amplified by a factor of 6 to 14 compared to unenhanced Raman signals through localized surface plasmon resonance (Lee et al. 2015; Nguyen et al. 2016; Willets 2009). However, applicability of SERS is limited due to the strong background signal when the concentration of complex clinical samples, especially target biomolecules, is very low (Li et al. 2013). Therefore, it is essential to develop a SERS biosensor having sufficiently high sensitivity and selectivity to use label-free SERS detection in clinical samples.

Thereamong, a 3D plasmonic nanostructure is capable of providing a higher SERS effect because an electric field incident on the 3D plasmonic nanostructure is greatly amplified at SERS active sites called electromagnetic “hotspots” through localized surface plasmon effects compared to 2D plasmonic substrates such as nanofilms or nanodots (Wang et al. 2015; Wang et al. 2018). This enhancement effect enables the recognition of Raman spectral fingerprints in individual molecules, overcoming difficulty of identifying various analytes and low sensitivity in conventional Raman signals (Lin et al. 2014; Wang et al. 2017). Hence, this unique property may be combined with molecular fingerprint specificity and single-molecule sensitivity to provide the ability to detect multiple miRNAs (Li et al. 2013).

Against this background, the present inventors constructed a 3D hierarchical nanobiosensor using self-assembled DNA-probe-conjugated gold nanoparticles (SAP-AuNPs) on a head-flocked gold nanopillar structure, and ascertained that this biosensor may be very effective for label-free quantitative detection of PC-associated exosomal miRNAs in urine samples, thus culminating in the present invention.

### SUMMARY OF THE INVENTION

An object of the present invention is to provide a surface-enhanced Raman scattering (SERS) biosensor for diagnosing prostate cancer with high sensitivity, including a SERS-based 3D hierarchical nanosubstrate with a SERS signal amplified, which includes a head-flocked metal nanopillar structure, a capture DNA probe specifically binding to a prostate-cancer-derived target biomarker linked to the heads of the metal nanopillar structure, and metal nanoparticles with a detection DNA probe conjugated thereto located in the nanogaps between the heads of the metal nanopillar structure; a Raman microscope to which the nanosubstrate is attached; and a measurement unit configured to measure the amplified Raman signal with the Raman microscope.

Another object of the present invention is to provide a method of detecting a prostate-cancer-derived target biomarker based on surface-enhanced Raman scattering (SERS), including detecting a prostate-cancer-derived target biomarker using the biosensor.

Still another object of the present invention is to provide a surface-enhanced Raman scattering (SERS) biosensor for detecting a prostate-cancer-derived target biomarker with high sensitivity, including a SERS-based 3D hierarchical nanosubstrate with a SERS signal amplified, which includes a head-flocked metal nanopillar structure, a capture DNA probe specifically binding to a prostate-cancer-derived target biomarker linked to the heads of the metal nanopillar structure, and metal nanoparticles with a detection DNA probe conjugated thereto located in the nanogaps between the heads of the metal nanopillar structure; a Raman microscope to which the nanosubstrate is attached; and a measurement unit configured to measure the amplified Raman signal with the Raman microscope.

In order to accomplish the above objects, the present invention provides a surface-enhanced Raman scattering (SERS) biosensor for diagnosing prostate cancer with high sensitivity, including a SERS-based 3D hierarchical nanosubstrate with a SERS signal amplified, which includes a head-flocked metal nanopillar structure, a capture probe specifically binding to a prostate-cancer-derived target biomarker linked to the heads of the metal nanopillar structure, and metal nanoparticles with a detection probe conjugated thereto located in the nanogaps between the heads of the metal nanopillar structure; a Raman microscope to which the nanosubstrate is attached; and a measurement unit configured to measure the amplified Raman signal with the Raman microscope.

In an embodiment of the present invention, the metal nanopillar structure may include a metal plate and metal nanopillars (heads).

In another embodiment of the present invention, the metal may be at least one selected from the group consisting of gold, silver, platinum, and aluminum.

In still another embodiment of the present invention, the head-flocked nanopillar structure may generate an enhanced SERS signal by reducing the distance between the nanopillar heads.

In yet another embodiment of the present invention, the metal nanoparticles may have a size of 7 to 50 nm.

In still yet another embodiment of the present invention, the nanogaps may have a size of 3 to 20 nm.

In even yet another embodiment of the present invention, the biomarker may be DNA, miRNA, or peptide.

In a further embodiment of the present invention, the miRNA may be miRNA-10a or miRNA-21.

In still a further embodiment of the present invention, the probe may be 75 bp to 150 bp long.

In yet a further embodiment of the present invention, the end of each of the capture probe and the detection probe may be modified with a thiol group.

In still yet a further embodiment of the present invention, the probe may include DNA or LNA.

In even yet a further embodiment of the present invention, the biosensor may detect an exosome-derived miRNA protein by measuring a change in Rayleigh scattering spectrum caused by specific binding of exosome-derived miRNA.

In even still a further embodiment of the present invention, the biosensor may detect a prostate-cancer-derived target biomarker in a wide range of attomolar concentration (aM) to nanomolar concentration (nM).

In addition, the present invention provides a method of detecting a prostate-cancer-derived target biomarker based on surface-enhanced Raman scattering (SERS), including detecting a prostate-cancer-derived target biomarker using the surface-enhanced Raman scattering (SERS) biosensor described above.

In addition, the present invention provides a surface-enhanced Raman scattering (SERS) biosensor for detecting a prostate-cancer-derived target biomarker with high sensitivity, including a SERS-based 3D hierarchical nanosubstrate with a SERS signal amplified, which includes a head-flocked metal nanopillar structure, a capture probe specifically binding to a prostate-cancer-derived target biomarker linked to the heads of the metal nanopillar structure, and metal nanoparticles with a detection probe conjugated thereto located in the nanogaps between the heads of the metal nanopillar structure; a Raman microscope to which the nanosubstrate is attached; and a measurement unit configured to measure the amplified Raman signal with the Raman microscope.

### BRIEF DESCRIPTION OF THE DRAWINGS

FIG. 1 schematically shows a biosensor according to the present invention;

(A) and (B) of FIG. 2 show results of measurement of UV-visible spectrum of 15 nm-sized metal nanoparticles (AuNPs) and metal nanoparticles (SAP-AuNPs) and XPS spectrum of SAP-AuNPs;

(A) to (E) of FIG. 3 show SEM images of hierarchical 3D SERS structures after self-assembly of head-flocked gold nanopillars without SAP-AuNPs (A) and metal nanoparticles (SAP-AuNPs) having respective sizes of 5 nm (B), 15 nm (C), 30 nm (D), and 50 nm (E);

(A) and (B) of FIG. 4 show SEM images of 15 nm-sized SAP-AuNPs with or without target miRNA;

(A) to (D) of FIG. 5 show Raman signals of miRNA located in the gaps greatly amplified by the plasmonic hotspots formed between the SAP-AuNPs and the nanopillar structure heads;

(A) to (D) of FIG. 6 show numerical simulation of the near-field distribution of hierarchical 3D SERS structures with SAP-AuNPs having respective sizes of 5 nm (A), 15 nm (B), 30 nm (C), and 50 nm (D) attached to nanopillars;

(A) to (D) of FIG. 7 show results confirming that the SERS sensor of the present invention based on a 3D hierarchical SERS substrate exhibits homogeneous SERS signal intensity in the spectrum and fingerprint peak in response to miRNA attachment to miR-10a and miR-21;

(A) and (B) of FIG. 8 show the relative standard deviation (RSD) values of characteristic peaks for miR-10a (1143 cm⁻¹) and miR-21 (1376 cm⁻¹);

(A) and (B) of FIG. 9 show Raman spectral changes of miR-10a (A) and miR-21 (B) under different treatment conditions;

(A) and (B) of FIG. 10 show results confirming that the 3D SERS biosensor of the present invention has specificity at the single nucleotide level without cross-reactivity and cross-hybridization with other miRNA sequences required for label-free detection of PC-associated exosomal miRNAs;

(A) to (C) of FIG. 11 show results confirming the SERS spectra of miRNA samples measured over the concentration gradient;

(A) to (C) of FIG. 12 show results of measurement of the SERS spectrum using the label-free 3D SERS biosensor of the present invention and qRT-PCR after extracting exosomal miRNAs from urine samples; and

(A) and (B) of FIG. 13 show sensitivity, specificity, and accuracy of diagnosis based on miR-10a (A) and miR-21 (B) using the 3D SERS sensor of the present invention.

### DETAILED DESCRIPTION OF THE INVENTION

The present invention is described in detail. The description and embodiments disclosed in the present invention may be applied to other descriptions and embodiments. Specifically, all combinations of various elements disclosed herein fall within the scope of the present invention. Also, the following description is not to be construed as limiting the scope of the present invention.

Moreover, those skilled in the art will be able to recognize or ascertain many equivalents to specific embodiments of the invention described herein using no more than routine experimentation. Also, such equivalents are intended to be encompassed by the present invention.

Hereinafter, a detailed description will be given of the present invention.

An aspect of the present invention pertains to a surface-enhanced Raman scattering (SERS) biosensor for diagnosing prostate cancer with high sensitivity, including a SERS-based 3D hierarchical nanosubstrate with a SERS signal amplified, which includes a head-flocked metal nanopillar structure, a capture probe specifically binding to a prostate-cancer-derived target biomarker linked to the heads of the metal nanopillar structure, and metal nanoparticles with a detection probe conjugated thereto located in the nanogaps between the heads of the metal nanopillar structure; a Raman microscope to which the nanosubstrate is attached; and a measurement unit configured to measure the amplified Raman signal with the Raman microscope.

As used herein, the term “biosensor” is a device for sensing using a special reaction between a biological material such as an enzyme and an antibody and a molecule to be detected in a complex mixture, and may be used interchangeably with immunosensor, and the biosensor of the present invention may be used to diagnose prostate cancer by detecting a prostate-cancer-derived target biomarker.

The biosensor may be SERS-based label-free.

As used herein, the term “SERS-based 3D hierarchical nanosubstrate with a SERS signal amplified” refers to a surface-enhanced Raman scattering (SERS)-based substrate configured to detect a prostate-cancer-derived target biomarker, including a head-flocked metal nanopillar structure, a capture probe specifically binding to a prostate-cancer-derived target biomarker linked to the heads of the metal nanopillar structure, and metal nanoparticles with a detection probe conjugated thereto located in the nanogaps between the heads of the metal nanopillar structure. The term “3D hierarchical nanosubstrate” refers to a nanosubstrate having a 3D hierarchical structure, configured such that a capture probe specifically binding to a prostate-cancer-derived target biomarker is bound to the heads of a head-flocked metal nanopillar structure and self-assembled metal nanoparticles with a detection probe conjugated thereto are located in the nanogaps between the heads of the nanopillar structure.

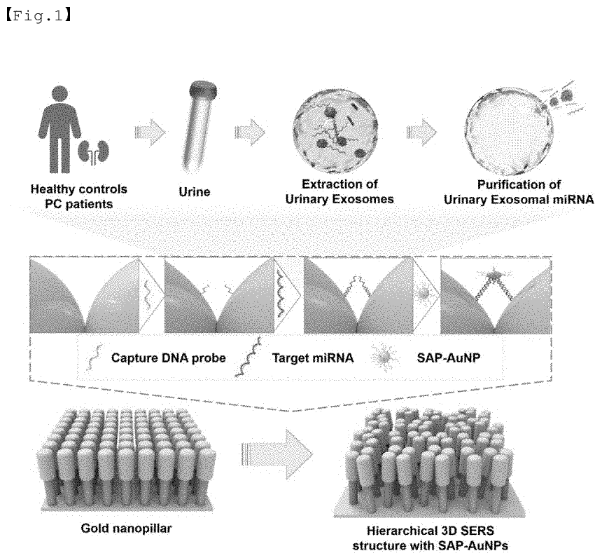

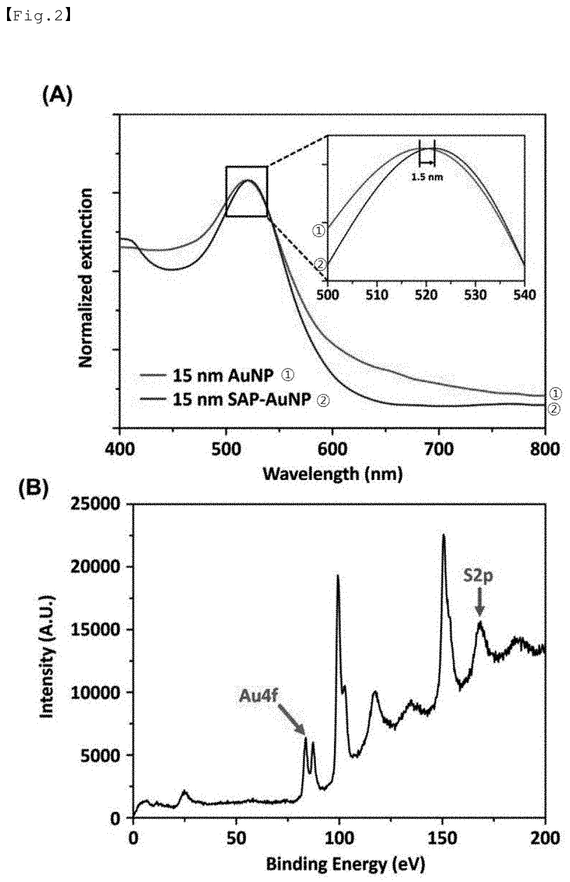

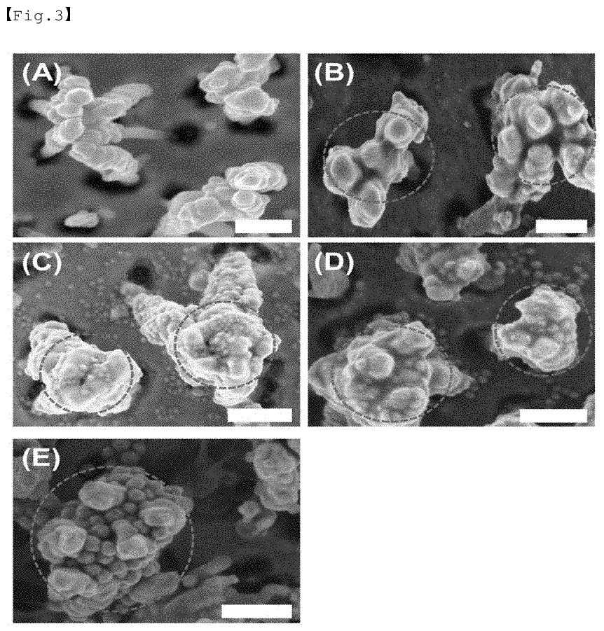

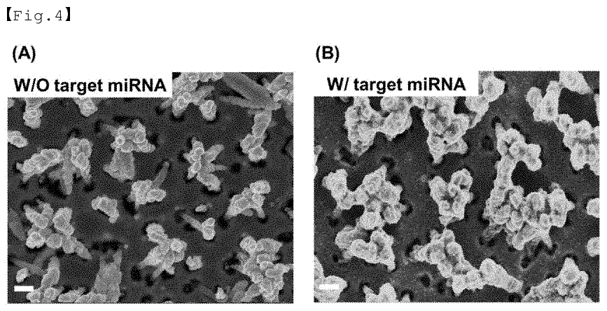

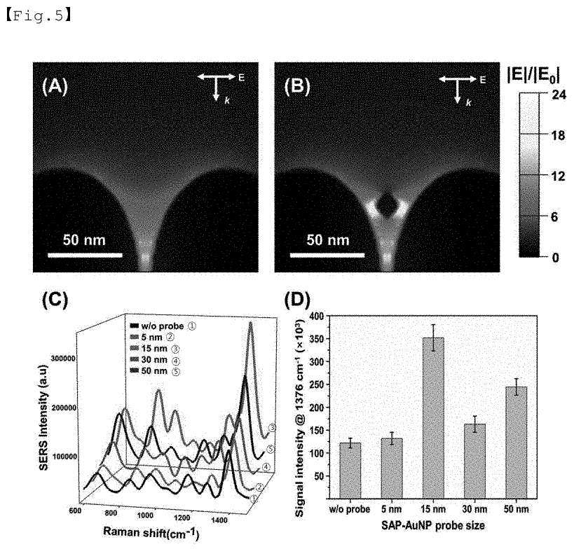

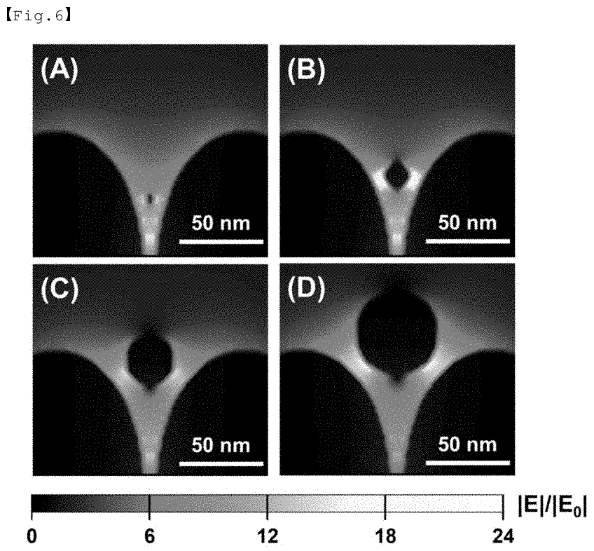

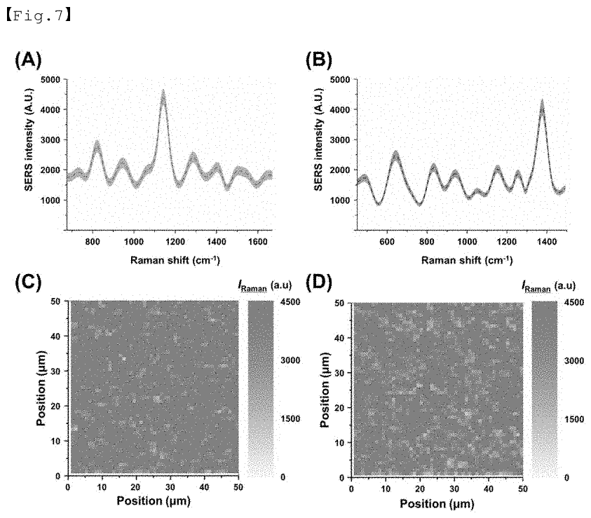

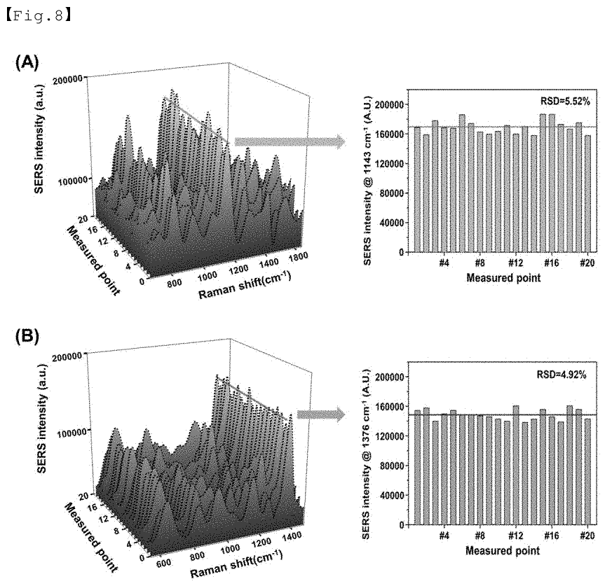

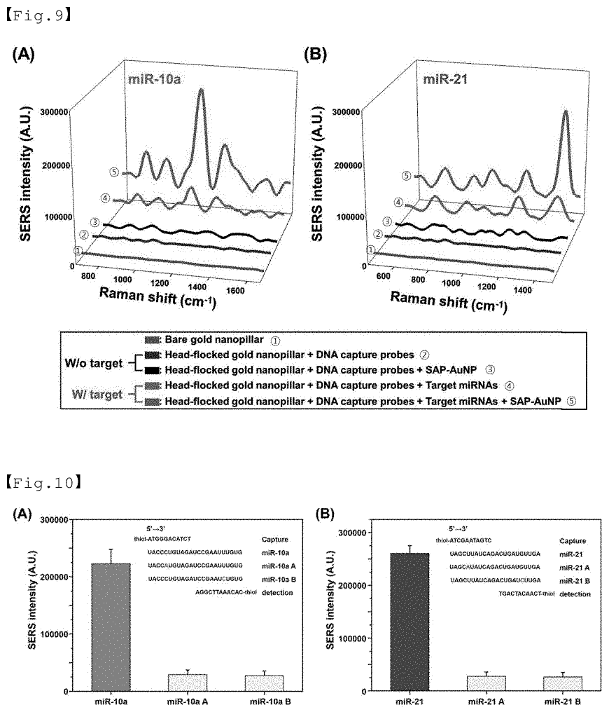

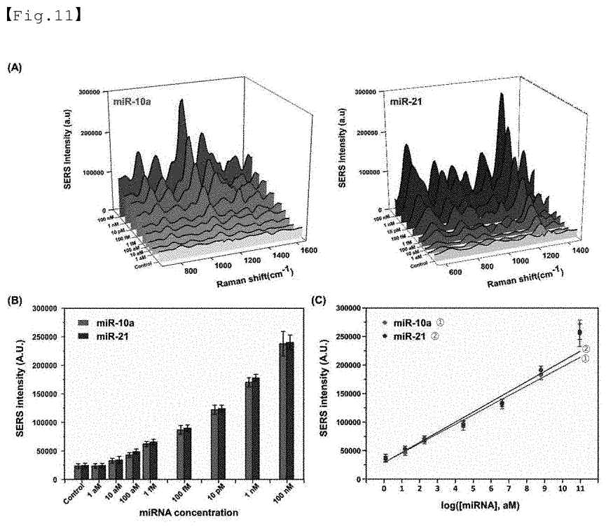

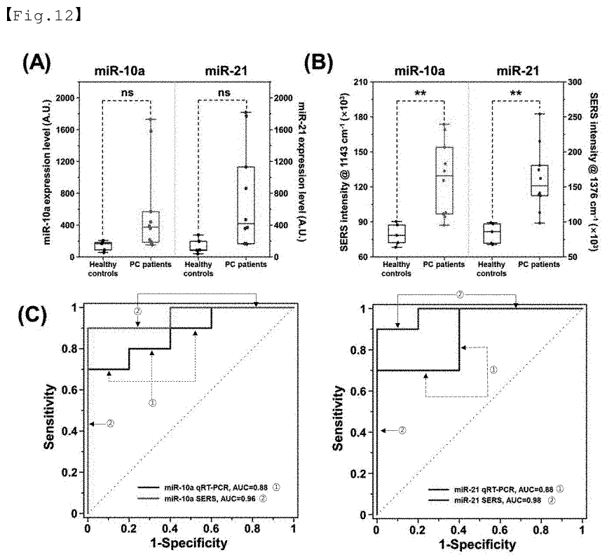

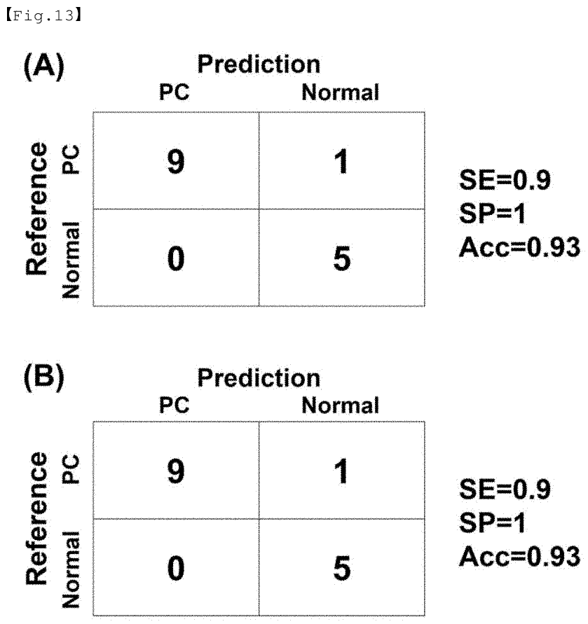

As used herein, the term “head-flocked metal nanopillar structure” refers to a structure including a thin metal plate that allows column-shaped metal nanopillars to be erected and metal nanopillars (metal heads) in the form of being erected thereon, and indicates a structure in which the heads of the metal nanopillars are gathered in order to measure a more amplified Raman signal. As schematically shown in FIG. 1, the structure may be in a form in which individual metal nanopillars are gathered by a capillary phenomenon.

The head-flocked nanopillar structure is capable of generating an enhanced SERS signal by decreasing the distance between the nanopillar heads.

The metal may be at least one selected from the group consisting of gold, silver, platinum, and aluminum, and particularly may be gold, but is not limited thereto.

A half capture probe specifically binding to the prostate-cancer-derived target biomarker may be linked to the heads of the metal nanopillar structure.

As used herein, the term “target biomarker” refers to a biomarker to be detected or diagnosed in an isolated form obtained in a living body, and may be, for example, DNA, miRNA, or peptide, and in another example may be miRNA, but is limited thereto.

Here, miRNA, particularly microRNA, is short (20-24 nt) non-coding RNA that is involved in post-transcriptional regulation of gene expression in multicellular organisms by affecting both stability and translation of mRNA. For example, miRNA may be derived from exosomes, and in another example may be exosome-derived miRNA obtained from a subject having prostate cancer.

Exosomes are a kind of extracellular vesicles (EVs) that are generated within cells and released to the outside, and are vesicles having a size of 50 to 150 nm that are secreted through information exchange between cells in eukaryotes.

The exosome-derived miRNA may be, but is not limited to, miRNA-10a, miRNA-21, or a combination thereof, and miRNA-10a and miRNA-21 may include sequences known in NCBI and the like.

The prostate-cancer-derived target biomarker may be included in an isolated sample of a subject having prostate cancer, such as plasma or urine, and particularly may be urine, but is not limited thereto.

“Capture probe” may be used interchangeably with binding probe, etc., and may specifically bind to a target or a target biomarker to be detected, and thereamong, a half capture probe indicates a probe having a sequence corresponding to half of a sequence that specifically binds to a prostate-cancer-derived target biomarker.

The capture probe may include DNA or LNA.

Here, LNA is introduced to further improve the sensitivity and selectivity of the sensor, and may include a moiety of a chimeric ribose ring fixed between 2′-oxygen and 4′-carbon through an O-methylene bridge. LNA thus configured is capable of improving hybridization specificity, duplex stability, and binding affinity of the biosensor.

The capture probe may be a probe specifically binding to a prostate-cancer-derived biomarker, for example, miRNA-10a or miRNA-21, and may be 75 bp to 150 bp in length, but is not limited thereto.

The metal nanoparticles may be located in the nanogaps between the heads of the metal nanopillar structure, and may be characterized in that a detection probe is conjugated thereto.

The metal nanoparticles may have a size of 7 to 50 nm, particularly 10 to 20 nm. When the particle size thereof falls in the above range, a vastly superior SERS response may appear compared to other nanoparticle sizes.

The nanogap is a nanometer-sized gap formed when the heads of the nanopillar structure are gathered, and the size thereof may be, for example, 3 to 20 nm, but is not limited thereto.

The metal may be any one selected from the group consisting of gold (Au), copper (Cu), platinum (Pt), and palladium (Pd), and may be, for example, gold, but is not limited thereto.

As used herein, the term “detection probe” refers to a probe specifically binding to a prostate-cancer-derived target biomarker, and this probe may be conjugated or bound to the metal nanoparticles.

The “prostate-cancer-derived target biomarker” is as described above.

The detection probe may specifically bind to a target or a target biomarker to be detected, and thereamong, a half detection probe indicates a probe having a sequence corresponding to half of a sequence that specifically binds to a prostate-cancer-derived target biomarker.

The detection probe may include DNA or LNA.

The end of each of the capture DNA probe and the detection DNA probe may be modified with a thiol group. This serves to effectively conjugate the capture probe to the heads of the metal nanopillar structure and the detection probe to the metal nanoparticles.

As used herein, the term “Raman microscope” refers to a device capable of analysis through Raman imaging by fixing the surface-enhanced Raman scattering (SERS)-based substrate for detecting a prostate-cancer-derived biomarker to a slide such as a glass slide, and the term “measurement unit configured to measure a Raman signal” refers to a device configured such that a biofluid such as plasma or urine is treated on the substrate and binds to a capture probe specifically binding to a prostate-cancer-derived target biomarker contained therein, followed by measurement of the Raman signal generated by a SERS signal tag.

The biosensor is capable of detecting an exosome-derived miRNA protein by measuring a change in Rayleigh scattering spectrum caused by specific binding of exosome-derived miRNA.

Also, the biosensor is capable of detecting exosome-derived miRNA in a very small amount ranging from attomolar concentration to nanomolar concentration.

According to an embodiment of the present invention, the biosensor is capable of detecting exosome-derived miRNA even at a low detection limit, but the present invention is not limited thereto.

Another aspect of the present invention pertains to a method of diagnosing prostate cancer with high sensitivity including treating a biomarker mixture with the biosensor.

Here, the terms “surface-enhanced Raman scattering (SERS)-based biosensor”, “biomarker”, and “SERS” are as described above.

As used herein, the term “prostate cancer” refers to cancer occurring in the prostate gland, and is mostly adenocarcinoma (cancer of gland cells) occurring in prostate cells. The types thereof are classified depending on the extent of differentiation of tumor tissue and the characteristics of cells. A widely useful classification method was suggested by a pathologist named Donald Gleason. It is also reported that prostate cancer is divided into grade 1, the best grade, to grade 5, the lowest, and the better the differentiation, the better the prognosis.

Still another aspect of the present invention pertains to a method of detecting a prostate-cancer-derived target biomarker based on surface-enhanced Raman scattering (SERS), including detecting a prostate-cancer-derived target biomarker using the surface-enhanced Raman scattering (SERS)-based biosensor.

Here, the terms “surface-enhanced Raman scattering (SERS)-based biosensor”, “biomarker”, and “SERS” are as described above.

The detection method may include detecting the prostate-cancer-derived target biomarker by measuring the intensity of Raman signals in an average wavelength range of 500 to 2000 cm⁻¹ using surface-enhanced Raman scattering (SERS).

More specifically, the detection method may include fixing the surface-enhanced Raman scattering (SERS)-based substrate for detecting a prostate-cancer-derived target biomarker to a Raman microscope, adding plasma or urine containing a target biomarker thereto, and collecting and processing Raman signals using a Raman signal measurement unit.

Yet another aspect of the present invention pertains to a method of diagnosing prostate cancer based on surface-enhanced Raman scattering (SERS), including detecting a prostate-cancer-derived target biomarker using the surface-enhanced Raman scattering (SERS)-based substrate for detecting a prostate-cancer-derived target biomarker, and diagnosing prostate cancer by identifying a Raman signal for the prostate-cancer-derived target biomarker.

Here, the terms “surface-enhanced Raman scattering (SERS)-based biosensor”, “biomarker”, and “SERS” are as described above.

Still yet another aspect of the present invention pertains to a surface-enhanced Raman scattering (SERS) biosensor for detecting a prostate-cancer-derived target biomarker with high sensitivity, including a SERS-based 3D hierarchical nanosubstrate with a SERS signal amplified, which includes a head-flocked metal nanopillar structure, a capture probe specifically binding to a prostate-cancer-derived target biomarker linked to the heads of the metal nanopillar structure, and metal nanoparticles with a detection probe conjugated thereto located in the nanogaps between the heads of the metal nanopillar structure; a Raman microscope to which the nanosubstrate is attached; and a measurement unit configured to measure the amplified Raman signal with the Raman microscope.

Here, the terms “surface-enhanced Raman scattering (SERS)-based biosensor”, “biomarker”, “SERS”, “capture probe”, “metal nanoparticles”, “detection probe”, “Raman microscope”, and “Raman signal measurement unit” are as described above.

A better understanding of the present invention may be obtained through the following examples. These examples are merely set forth to illustrate the present invention and are not to be construed as limiting the scope of the present invention.

### Experimental Example 1: Chemicals and Materials

Unless otherwise specified, all chemicals used in this experiment were purchased from Sigma-Aldrich, Korea. In addition, Au pellets (Ø 3×3 mm, 99.99%) were purchased from Shinwoo Metal (Korea). 1.5 mL DNA LoBind tubes were provided by Eppendorf. Ambion® nuclease-free water was purchased from Thermo Fisher Scientific Korea. All solutions were prepared with deionized water (18.2 mΩ/cm). DNA probes and target miRNA sequences were provided by Bioneer (Korea). Total exosomal RNA isolation kit (Invitrogen™) was purchased from Thermo Fisher Scientific, MA, USA.

### Experimental Example 2: Preparation of Hierarchical 3D SERS Substrate

First, a head-flocked gold nanopillar structure was constructed through maskless reactive ion etching and electron beam evaporation as previously known (Lee et al. 2019). Specifically, a silicon nanopillar mold was manufactured using a maskless RIE process, which is dry etching using a chemically reactive plasma without a film mask, and specific conditions were as follows: Plasmalab 100 system (UK, Oxford) Platen power of 110 W, SF6-to-O2 flow ratio of 1.12, speed of 3 nm/sec, and chamber pressure of 36 mTorr.

After the RIE process, silicon surface residue was physically removed by exposure to oxygen plasma in order to prevent the influence thereof on the SERS spectrum. Thereafter, in order to metallize the silicon mold, gold was deposited on the surface of the silicon nanopillars through electron beam evaporation, and the silicon nanopillar substrate was further coated with gold. As shown in FIG. 1, a plasmonic head-flocking phenomenon could be confirmed during treatment of the gold nanopillars with the solution (FIG. 1).

Next, in order to fully conjugate the DNA probe to the nanostructure, the thiol group of the probe was activated using DTT according to the manufacturer's protocol. The activated capture probe (1 μM) was mixed with a conjugation buffer containing sodium sulfate (30 mM), after which the substrate was incubated with the mixture at 30° C. for 12 hours. After incubation, the substrate was washed with deionized water and dried through N2 blowing. Moreover, in order to prevent non-specific adsorption of other nucleic acids to the gold surface, the nanopillar substrate was treated with a monolayer of 6-mercapto-1-hexanol (MCH). The probe-bound substrate was incubated in an MCH solution (10 μM) at room temperature for 3 hours and then washed with a wash buffer containing 2× saline-sodium citrate (SSC) and 0.2% SDS. Thereafter, the substrate was thoroughly washed once more and dried with N2. Next, in order to evaluate the sensitivity or specificity of the sensor, a sample was prepared by adding target miRNA to human serum at a concentration ranging from 1 aM to 1 μM. Thereafter, the substrate was incubated at 30° C. for 12 hours, washed, and then dried with N2. Finally, in order to hybridize SAP-AuNPs with miRNA, the substrate was incubated with a hybridization buffer containing 2×SSC and SAP-AuNPs (10 nM) in nuclease-free water at 30° C. for 6 hours, followed by DI washing and N2 drying for SERS measurement.

### Experimental Example 3: Numerical Simulation of Hierarchical 3D SERS Nanosensor

Numerical modeling and simulation of the nanosensor were performed using Lumerical FDTD simulation (Lumerical Inc.), and dimensional information of the sensor was calculated using a GeoGebra graphic calculator. Specifically, the simulation was performed with SAP-AuNPs having various sizes disposed in the gaps between the nanopillar heads, and two silicon nanopillars with gold deposited on the oval top thereof were disposed at a distance of 250 nm. After wetting, the nanopillars were slightly tilted toward each other due to capillary force. The distance between the gold nanopillar heads was set to 10 nm. A DNA probe was attached to the gaps between the heads of the nanopillar structure by complementary hybridization with target miRNA. In this simulation, four different sizes (5, 15, 30, and 50 nm) of SAP-AuNPs were confirmed, and the distance between the SAP-AuNPs and the nanopillar structure heads was fixed at 8 nm, which is the same size as miRNA. Polarized plane-wave light at a wavelength of 785 nm was allowed to be directly incident on the nanopillar structure. The simulation mesh size was set to 1 nm and the frequency of incident light was 3.82×10¹⁴ Hz (Ding et al. 2016).

### Experimental Example 4: DNA Probe Design and SAP-AuNP Synthesis

Each of half-complementary DNA probes for miRNA capture and detection was designed to specifically bind to target miRNA. First, a capture DNA probe was bound to the head-flocked nanopillar structure, and a detection probe was attached to gold nanoparticles (AuNPs). Here, in order to prevent overlapping of three or more bases in the DNA probe, the Tm value for optimal hybridization was calculated using the OligoAnalyzer® Tool (Integrated DNA Technologies, USA). Sequence information on the target miRNA and the corresponding DNA probe is shown in Table 1 below.

TABLE 1
Sequences of DNA probe and target miRNA

| miRNA designation | Sequence (5′→3′) | Length (nt) |
|---|---|---|
| miR-21 | UAGCUUAUCAGACUGAUGUUGA | 22 |
| miR-21 A | UAGCAUAUCAGACUGAUGUUGA | 22 |
| miR-21 B | UAGCUUAUCAGACUGAUGUUGA | 22 |
| miR-10a | UACCCUGUAGAUCCGAAUUUGUG | 23 |
| miR-10a A | UACCAUGUAGAUCCGAAUUUGUG | 23 |
| miR-10a B | UACCCUGUAGAUCCGAAUCUGUG | 23 |
| Probe DNA | | |
| designation | Sequence (5′→3′) | Length (nt) |
| miR-10a capture | TCTACAGGGTA-C6-thiol | 11 |
| probe | | |
| miR-10a detection | thiol-C6-CACAAATTCGGA | 12 |
| probe | | |
| miR-21 capture | CTGATAAGCTA-C6-thiol | 11 |
| probe | | |
| miR-21 detection | thiol-C6-TCAACATCAGT | 11 |
| probe | | |

In order to conjugate respective DNA probes to the gold nanopillar structure and the AuNPs, each of the 5′-end of the capture probe and the 3′-end of the detection probe was modified with a thiol group.

AuNPs for the preparation of SAP-AuNPs were synthesized through chemical reduction of a HAuCl₄ solution using trisodium citrate (Turkevich et al. 1951). Specifically, before conjugation of the DNA detection probe to NPs, the modified DNA detection probe was activated so as to inactivate the thiol by incubating 10 μL of a 1.0 N 1,4-dithiothreitol (DTT) solution for 15 minutes at room temperature according to the manufacturer's protocol. Thereafter, excess DTT and thiol-fragment were removed through three extraction processes with ethyl acetate (50 μL). At the same time, citrate-AuNPs were exchanged with bis(p-sulfonatophenyl)phenylphosphine dihydrate dipotassium (BSPP) with gentle stirring after adding a 0.1 M BSPP solution to the citrate-AuNP colloid. Excess BSPP was centrifuged and AuNPs were washed with deionized water. Next, a mixture of the detection DNA probe and the BSPP-coated AuNPs (110:1) was incubated overnight at room temperature for conjugation of AuNPs with the thiol group of DNA (Hill et al. 2009). Thereafter, the incubated solution was centrifuged at 6000 rpm for 10 minutes at room temperature to separate the unbound detection DNA probe. The detection DNA probe-AuNP solution was buffered to a pH of 7 with 10 mM PB, 0.01% sodium dodecyl sulfate (SDS), and 1.0 M NaCl. Thereafter, the SAP-AuNPs thus synthesized were analyzed with a UV-visible spectrophotometer (Shimadzu UV-3600, Japan) and an X-ray photoelectron spectrometer (ULVAC-PHI X-TOOL, Japan).

### Experimental Example 5: Isolation of Exosomes from Human Urine and Purification of Exosomal miRNA

Human urine samples used in the present invention were obtained from Korea University Anam Hospital (Seoul, Korea). The protocol of the clinical study was approved by the Institutional Review Board (IRB) of Korea University Anam Hospital (IRB No. 2016AN0287). In addition, all subjects recruited in the present invention signed an IRB-approved consent form. Urinary exosomes were isolated according to a previously reported protocol (Fernandez-Llama et al. 2010). Specifically, human urine samples (30 mL) were collected from prostate cancer (PC) patients and healthy controls, followed by centrifugation at 200×g for 10 minutes at 4° C. and then at 2000×g for 20 minutes at 4° C., and as such, cells, cell debris, and microvesicles were discarded. Cell-free urine was obtained, after which this sample was further centrifuged with DTT (200 mg/mL) at 17000×g for 10 minutes at 37° C. to remove urine proteins. The supernatant containing exosomes was concentrated to a final volume of 500 μL using a 15 mL centrifugal filter with a 10 kDa molecular weight cut-off membrane (Amicon Ultra, Merck Millipore, USA). Thereafter, exosomes were isolated from the concentrated supernatant using size exclusion chromatography (qEV column, Izon Science Ltd., New Zealand). In addition, sample elution was performed using phosphate-buffered saline (PBS) in 0.5 mL fractions according to the manufacturer's protocol, and fractions 7 to 11 containing the concentrated exosomes were collected. The eluate was further concentrated to a final volume of about 200 μL using a 0.5 mL centrifugal filter (Amicon Ultra, Merck Millipore). Then, exosomal miRNA was extracted from the concentrated exosomes using the Total Exosome RNA and Protein Isolation Kit (Invitrogen, CA, USA) according to the protocol provided by the manufacturer. The purified solution (10 μL) containing miRNA was diluted to 200 μL with 5×SSC.

### Experimental Example 6: Exosomal miRNA Detection Using 3D SERS Biosensor

After incubation of the sample containing miRNA on the SERS substrate, the substrate was fixed on a slide glass with silicone gel. The SERS signal of miRNA was measured with a custom Raman microscope (NOST, Korea). A 785 nm wavelength excitation laser was focused with an objective lens (TU Plan ELWD 100× air objective, Nikon, Japan; numerical aperture: 0.6, working distance: 0.56 mm) provided to an upright microscope (Eclipse Ni-U, Nikon). The laser power was adjusted to 10.67 mW with a PM100 power meter (Thorlabs, USA). The signal was generated by exposing the sample to the laser for a 0.5 sec exposure time in 30 accumulations. The confocal slit size and ND (Neutral Density) filter ratio were set to 120 μm and 16%, respectively. Raman signals were processed using a FEX spectrometer (NOST) connected to a Newton 920 charge-coupled device (CCD) camera (Andor Technology, UK). Raman mapping for each mapping measurement was performed on a 50×50 μm² square area with a 1 μm step size. The acquired data was analyzed using RAON-Vu software (NOST), Sigmaplot 10 software (Systat Software Inc., CA, USA), and Origin 8 software (OriginLab, MA, USA).

### Experimental Example 7: Quantitative Reverse Transcription Polymerase Chain Reaction (qRT-PCR) Analysis

The expression levels of urinary exosomal miRNA in clinical samples were quantified through qRT-PCR. Reverse transcription of total RNA was performed in urinary exosomes (1-10 ng, 15 μL of reaction volume) with the TaqMan miRNA reverse transcription kit (Applied Biosystems, USA) under optimized manufacturer conditions. Real-time qPCR was performed using the TaqMan miRNA assay kit with TaqMan Universal PCR master mix II (2×, Applied Biosystems) in the Step One Plus real-time PCR system (Applied Biosystems). Here, 40 cycles of 95° C. for 10 minutes, 95° C. for 15 seconds, and 60° C. for 1 minute as step cycle conditions were successively conducted. Thereafter, the cycle threshold (Ct) value of miRNA was determined using SDS software (v2.0.1; Applied Biosystems), and the relative expression level of miRNA was normalized by endogenous control RNA (U6 RNA) using the 2-ΔΔCt method (Folini et al. 2010).

### Example 1: Preparation and Characterization of Hierarchical 3D Plasmonic Nanopillar Structure and SERS Biosensor

In order to ensure sufficient sensitivity for clinical-level detection of exosomal miRNA with SERS, it is essential to construct a plasmonic nanopillar structure having high-density plasmonic hotspots to generate strong near-field amplification. To this end, a SERS biosensor was constructed using self-assembled DNA-probe-conjugated gold nanoparticles (SAP-AuNPs) on a plasmonic head-flocked gold nanopillar SERS substrate in the presence of target urinary exosomal miRNA, thereby creating a large number of 3D hotspots inducing strong electromagnetic-field amplification.

First, according to previous studies, a plasmonic gold nanopillar structure was constructed through maskless reactive ion etching of a silicon wafer and electron beam evaporation (Kim et al. 2019b; Lee et al. 2019). When this elastic nanopillar structure was in the Wenzel state (i.e. completely wetted in solvent), elastic-capillary force generated between nanopillars drove the nanopillar heads to form and gather plasmonic hotspots across the entire surface of the substrate (Wang et al. 2018). In order to increase the sensitivity of the SERS sensor and improve clinical applicability, SAP-AuNPs were additionally applied thereto to form a hierarchical plasmonic nanostructure capable of extending the plasmonic hotspots into the 3D space between periodic nanopillars and SAP-AuNPs. After formation of the hierarchical nanostructure in this way, in order to detect miRNA, target miRNA was recognized using a sandwich hybridization method, particularly using a capture probe and a detection probe as two half-complementary DNA probes, such that errors were reduced by allowing target sequences to be recognized in a complex biofluid environment containing various materials. Interfering biomolecules (Afzalinia and Mirzaee 2020; Goldman et al. 2013; Kim et al. 2019a). When target miRNA was present, SAP-AuNPs functionalized with a half-complementary DNA detection probe strand trapped the target miRNA in the space between the AuNPs and the nanopillar heads, thus forming 3D hotspots.

First, conjugation of the detection DNA probe to AuNPs was confirmed based on shift of the UV-visible spectrum and peak information of X-ray photoelectron spectroscopy (XPS). As shown in (A) and (B) of FIG. 2, the DNA probe having a thiol group at the end thereof was immobilized to AuNPs through Au—S bonding to form SAP-AuNPs. As the detection probe was bound to the AuNP surface, the UV-visible spectrum was redshifted from 519 nm to 520.5 nm (A). Moreover, the presence of Au4f and S2p peaks in the XPS spectrum of SAP-AuNPs means that DNA was immobilized on AuNPs to generate Au—S bonds (B). These results indicate that SAP-AuNPs for identifying miRNA were successfully synthesized.

Meanwhile, 3D finite-difference time-domain (FDTD) simulation was performed to confirm the enhancement effect by SAP-AuNPs in the hierarchical nanostructure. As shown in FIG. 1, based on results of observation with a field-emission scanning electron microscope (FE-SEM), it was confirmed that a 3D hierarchical structure was formed through hybridization of target miRNA, a capture DNA probe, and SAP-AuNPs in the head-flocked gold nanopillar structure (FIGS. 1, (A) to (E) of FIG. 3, and (A) and (B) of FIG. 4). As seen in these SEM images, it was confirmed that, in the structure of the present invention, two nanopillars were tilted toward each other and one SAP-AuNP was disposed in the gap between the nanopillar heads. Consequently, it was found that the Raman signal of miRNA located in the gaps was greatly amplified by the plasmonic hotspots formed between the SAP-AuNPs and the heads of the nanopillar structure ((A) and (B) of FIG. 5) (Zhang et al. 2018). In particular, the effect of the SAP-AuNP size on the electric-field amplification was confirmed with SAP-AuNPs having four sizes (5, 15, 30, and 50 nm). As shown in (A) to (D) of FIG. 6, based on the simulation results, 15 nm-sized SAP-AuNPs were confirmed to induce the strongest near-field enhancement in the gaps ((A) to (D) of FIG. 5 and (A) to (D) of FIG. 6). Also, 15 nm-sized SAP-AuNPs were confirmed to have a 4-fold stronger SERS enhancement effect than a bare head-flocked nanopillar structure ((A) to (D) of FIG. 5 and (A) to (D) of FIG. 6). This is due to the surface curvature of the nanoparticles, which decreases convexity of the particle surface with an increase in the AuNP size, thus reducing the absorption or inelastic scattering of light to the nanoparticles, thereby weakening the surface electromagnetic field and lowering overall SERS intensity. In contrast, for 5 nm-sized SAP-AuNPs, the size of AuNPs was very small compared to the gaps between the AuNPs and the nanopillar heads (about 8 nm, corresponding to the 22-nucleotide miRNA length), confirming the absence of the near-field amplification induced by the plasmonic binding effect. Based on these results, it was found that SAP-AuNPs greatly contributed to the improvement of SERS signals, thereby increasing the sensitivity for miRNA detection in the plasmonic nanoarchitecture.

In addition, in order to evaluate validity of the simulation, experimental measurement of the SERS signal was performed using miR-21. First, the Raman intensity at the 1376 cm⁻¹ peak unique to miR-21 was compared in various configurations of the hierarchical nanoarchitecture having different nanoparticle sizes. Consequently, when SAP-AuNPs having various sizes were added thereto, signals were improved based on the trend shown in the simulation results, confirming that the strongest signal was generated from 15 nm-sized SAP-AuNPs. In fact, it was confirmed that 15 nm-sized SAP-AuNPs exhibited a 2.9-fold stronger signal enhancement than the head-flocked nanopillar structure without SAP-AuNPs. These results showed that the 3D plasmonic hotspots induced in the hierarchical nanostructure of the present invention amplified a stronger SERS signal, and also that this signal amplification made possible label-free direct detection of target biomarkers including miRNAs present in extremely trace amounts in biofluids.

### Example 2: Evaluation of miRNA Sensing Performance of SERS Biosensor of the Present Invention

Although the sensitivity of SERS was increased over time, problems such as low reproducibility and uniformity occurred in quantification of analytes (Jarvis et al. 2008). Hence, in order to utilize the 3D SERS sensor of the present invention based on the hierarchical structure in miRNA quantification, uniformity of the sensor was evaluated using two PC-associated miRNAs (miR-10a and miR-21). As shown in (A) and (B) of FIG. 8, the 3D SERS nanostructure-based sensor of the present invention exhibited high signal uniformity across the mapping area (2500 points) of the substrate for two miRNAs at 100 nM with a small standard deviation (SD). In particular, the SERS spectra showed different peak intensities because the two miRNAs have different sequences and base ratios. It was confirmed that miR-10a consistently produced a strong peak at 1143 cm⁻¹, and also that miR-21 showed a characteristic peak at 1376 cm⁻¹ for adenine CH₃ modification (Kim et al. 2019b). Since this also appears in the normal Raman spectrum, it was selected as a representative fingerprint peak of each miRNA. Table 2 below shows the SERS fingerprint peaks for miR-10a and miR-21.

TABLE 2
SERS fingerprint peaks for miR-10a and miR-21

| Target miRNA | Wavenumber (cm⁻¹) | Assignment |
|---|---|---|
| miR-10a | 825 | Adenine ring deformation |
| | 930 | Ribose vibration |
| | 1143 | Adenine C—N vibration |
| | 1290 | Cytosine C—N vibration |
| | 1410 | Adenine ring stretching |
| | 1490 | Guanine ring stretching |
| miR-21 | 730 | Adenine ring breathing |
| | 880 | Adenine ring breathing |
| | 995 | Guanine ring stretching |
| | 1240 | Cytosine/Uracil ring |
| | | stretching |
| | 1376 | Adenine ring stretching/C—H |
| | | bending |

The relative standard deviation (RSD) values of the characteristic peaks for miR-10a (1143 cm⁻¹) and miR-21 (1376 cm⁻¹) were 5.52 and 4.92%, respectively ((A) and (B) of FIG. 8). The signal uniformity was further confirmed by mapping of the Raman intensity in a square area of 50 μm×50 μm and the laser spot size of 1 μm diameter on the surface of the SERS sensor of the present invention ((C) and (D) of FIG. 7). The SERS sensor of the present invention based on the 3D hierarchical SERS substrate exhibited homogeneous SERS signal intensity in the spectrum and fingerprint peak in response to miRNA attachment to miR-10a and miR-21. Moreover, due to the Raman activity of the background SERS signal and the DNA probe of the 3D SERS platform without target miRNA, no disturbance to the SERS spectrum of target miRNA was confirmed ((A) and (B) of FIG. 9). These results indicate that the SERS sensor of the present invention generates a uniform and reproducible SERS signal for miRNA detection, which is necessary to have accurate and reliable diagnostic results.

Meanwhile, it is estimated that there are 2,300 miRNAs in the human body (Alles et al. 2019), some of which are classified as homologous miRNA family groups, and it is reported that there is a difference in only one or two nucleotides between members of the family groups (Dong et al. 2014, Garcia-Rico et al. 2018, Ye et al. 2019). Therefore, due to high sequence homology between these miRNAs, miRNA biosensors for application to disease diagnosis require specific discrimination of miRNAs with single nucleotide differences. In order to evaluate specificity of the SERS biosensor of the present invention in this respect, SERS signal intensities at signal intensities of perfectly matched target miRNAs were compared with two different single-base-mismatched miRNAs (type A and type B). Specifically, as is apparent from Table 1, type A miRNA had a single mismatch site in a sequence complementary to the detection probe, whereas type B miRNA had a single mismatch in a sequence complementary to the capture probe (Table 1). The SERS signal intensities of 1143 cm⁻¹ for miR-10a and 1376 cm⁻¹ for miR-21 were measured and compared for complete match with single-base-mismatched miRNAs ((A) and (B) of FIG. 10). As shown in (A) and (B) of FIG. 10, intense SERS signals at the same concentration (100 nM) were obtained only from perfectly matched target miRNAs, whereas all single-base-mismatched miRNAs (both types A and B) did not show recognizable SERS signals. These results indicate that the 3D SERS biosensor of the present invention had specificity at the single nucleotide level without cross-reactivity and cross-hybridization with other miRNA sequences required for label-free detection of PC-associated exosomal miRNAs.

In addition, since exosomes are present in very small amounts in body fluids, sensors to detect exosomal miRNAs require very high sensitivity, ideally below the femoral detection limit (Blondal et al. 2013; Max et al. 2018; Wang et al. 2020). Therefore, the detection limit and dynamic detection range of the 3D SERS biosensor of the present invention targeting PC-associated miRNA in a biofluid environment were confirmed. Specifically, each target miRNA sample was prepared through serial dilution from 100 nM to 1 aM in human serum, and a non-specific miRNA sequence was constructed as a control (Table 1). Then, the SERS spectra of the miRNA samples measured over the concentration gradient are shown in (A) to (C) of FIG. 11. As shown in (A) to (C) of FIG. 11, the signal at the SERS fingerprint peak of each miRNA was detected even at a deficient concentration (about 10 aM), based on which it was found that the 3D SERS biosensor of the present invention had a very low detection limit for target miRNA. In particular, it was confirmed that the fingerprint peak intensity for each of miR-10a and miR-21 had a good linear correlation over a wide concentration range from 10 aM to 100 nM. In order to confirm a quantitative correlation between the miRNA concentration and the SERS intensity, a linear regression of miRNA-specific peak intensities was plotted depending on target miRNA concentrations. As such, the linear regression equations were as follows.

y=22920 log x+1529.6,R²=0.968 for miR-10a

y=23039 log x+5118.1,R²=0.970 for miR-21

Here, x is the miRNA concentration.

All linear regressions for target miRNA showed a good correlation (R2>0.96) between the representative SERS peak intensity and the logarithm of the miRNA concentration. The detection limits evaluated were low for both miR-10a and miR-21 without chemical labeling. Compared to previously reported label-free SERS sensors (Kim et al. 2019b) and other amplification methods including fluorescent or electrochemical sensors (Chen et al. 2020; Li et al. 2020; Liu et al. 2020; Wan et al. 2020), Zhang et al. 2017), the 3D SERS biosensor of the present invention was confirmed to show at least 1000-fold lower detection limit (Table 3 below).

TABLE 3
Comparison of detection limits with
other literature on miRNA detection

| | | Detection | | |
|---|---|---|---|---|
| Detection method | Target | limit | | Ref. |
| Fluorescence | let-7a | 70.9 | fM | Chen et al., |
| | | | | 2020 |
| Fluorescence | miR-21 | 55 | fM | Li et al., |
| | | | | 2020 |
| Electrochemiluminescence | let-7a | 120 | fM | Liu et al., |
| | | | | 2020 |
| Electrochemical | miR-486-5p | 10 | fM | Wan et al., |
| | | | | 2020 |
| SERS | miR-141 | 100 | fM | Zhang et al., |
| | | | | 2017 |
| SERS | miR-10b, 21, 373 | 3.53, 2.17, | | Kim et al., |
| | | 2.16 fM | | 2019b |
| SERS | miR-10a, 21 | 10 | aM | This work |

The excellent detection limit of the biosensor of the present invention can be interpreted as originating from outstanding electric-field enhancement induced in the 3D plasmonic hotspots and confinement of target miRNA inside the 3D hotspots of the hierarchical nanoarchitecture. Therefore, taking into consideration the rarity of urinary exosomal miRNAs in the urine and the large difference in concentration between PC patients and healthy persons (Bryzgunova et al. 2016), it is suggested that the SERS sensor of the present invention having a very low detection limit and a wide dynamic range is suitable for the detection of urinary exosomal miRNA for diagnostic applications.

### Example 3: Clinical Distinction of PC Patients in Urine Samples

Urinary exosomes from PC patients contain unique PC-specific biomarkers, including miRNAs, in which exosomal miRNAs are particularly important biomarkers for early-stage diagnosis of PC and personalized approaches to patient prognosis and treatment. Despite growing interest in urinary exosomal miRNAs, the development of practical urinary exosomal miRNA-based biosensors for PC diagnosis is still challenging due to several important drawbacks, such as the complex composition of urine and low concentrations of exosomes (Leung et al. 2021, Li et al. 2019).

In order to confirm the potential for PC diagnosis, the label-free 3D SERS system of the present invention was used to quantify exosomal miRNAs in clinical urine samples obtained from 10 PC patients and 5 healthy controls (Table 4).

TABLE 4
Summary of clinical features of urine samples

| | | Healthy | |
|---|---|---|---|
| | | controls | PC patients |
| Characteristics | Category | (n = 5) | (n = 10) |
| Age (years) | ≤30 | 3 (60%) | — |
| | 31-69 | 2 (40%) | 3 (30%) |
| | ≥70 | — | 7 (70%) |
| | Mean ± SD | 29.6 ± 1.9 | 73.3 ± 7.0 |
| Prostate-specific | ≤4.0 | 4 (80%) | 5 (50%) |
| antigen (ng/mL) | 4.1-10.0 | 1 (20%) | 1 (10%) |
| | 10.1-20.0 | — | 1 (10%) |
| | ≥20.0 | — | 2 (20%) |
| | Mean ± SD | 1.84 ± 1.59 | 37.55 ± 90.04 |
| Gleason score | <7 | — | 1 (10%) |
| | ≥7 | — | 7 (70%) |
| | Not available | — | 2 (20%) |
| Cancer stage | IV | — | 10 (100%) |

Thereafter, based on results of analysis of surface protein expression and morphological images of purified exosomes released from tumor cells into urine, the confirmed transmission electron microscope images showed that the urinary exosomes were spherical with a diameter of 30-100 nm. Western blotting of purified exosomes showed that exosome-associated protein markers (ALIX and CD9) were present in urinary exosomes of both PC patients and healthy controls, whereas a non-exosome marker (calnexin) was absent.

In addition, the results of measurement of SERS spectrum with the label-free 3D SERS biosensor of the present invention and qRT-PCR after extracting exosomal miRNAs from urine samples are shown in (A) to (C) of FIG. 12. (A) to (C) of FIG. 12 are represented as box plots. As seen in (A) to (C) of FIG. 12, based on the results of measurement by qRT-PCR and the 3D SERS sensor, the expression levels of target miRNAs were higher in the PC patients than in the healthy controls. These results are consistent with literature reporting increased concentrations of both miRNAs in PC (Foj et al. 2017; Worst et al. 2020). Based on the results of calculation of p-values to assess statistically significant differences in exosomal miRNA expression levels between the two clinical groups, both qRT-PCR and the 3D SERS sensor of the present invention showed the same trend between miRNA concentration and PC generation, and qRT-PCR had a p-value greater than 0.01 for both miRNAs (miR-10a: 0.12, miR-21: 0.067), whereas the p-value of the label-free SERS 3D biosensor of the present invention was less than 0.01 (miR-10a: 0.0068, miR-21: 0.0035). Given that lower p-values have more statistically significant differences between groups (Berkson 2003; Lenzi et al. 2021), these results suggest that the 3D SERS sensor of the present invention shows the relationship between miRNA concentration and PC generation more clearly than qRT-PCR and has higher clinical reliability for disease diagnosis.

In addition, clinical sensitivity and selectivity of PC diagnosis using the 3D SERS biosensor of the present invention were evaluated through ROC (receiver operating characteristic) plotting. In the ROC graph, AUC (area under the curve) indicates diagnostic performance, in which non-critical diagnostic methods have an AUC close to 0.5 and a value of 1 indicates perfect discrimination between positive and negative samples (Berrar and Flach 2012). As shown in (C) of FIG. 12, in the ROC curves of qRT-PCR and the SERS biosensor of the present invention for PC diagnosis using miR-10a and miR-21, the 3D SERS biosensor of the present invention can be confirmed to have excellent sensitivity and selectivity for the diagnosis of PC patients. AUC of the 3D SERS biosensor of the present invention for target miRNAs was 0.96 (95% confidence interval (CI): 0.87-1.00) for miR-10a and 0.98 (95% CI: 0.91-1.00) for miR-21. In contrast, AUC of qRT-PCR was 0.88 (95% CI: 0.70-1.00) for miR-10a and 0.88 (95% CI: 0.71-1.00) for miR-21. In conclusion, the miRNA-based PC diagnosis using the 3D SERS biosensor of the present invention had an accuracy of 0.93 for both miR-10a and miR-21 ((A) and (B) of FIG. 13), which is evaluated to be much higher than the accuracy of serum PSA-based PC diagnosis reported in other literature. Therefore, these results demonstrate that the label-free 3D SERS biosensor using the hierarchical SERS substrate of the present invention has excellent clinical potential in diagnosing PC by effectively detecting exosome-derived urinary miRNAs.

According to the present invention, the SERS-based label-free biosensor is configured such that a 3D hierarchical nanoarchitecture is constructed through self-assembly of a plasmonic head-flocked gold nanopillar SERS substrate and SAP-AuNPs in the presence of target miRNA, and uniform and rich nanogaps of the 3D nanoarchitecture thus constructed lead to intense broadening of the intrinsic SERS fingerprint peak of target miRNA, resulting in ultrahigh sensitivity (about 10 aM) to the target miRNA and specific detection performance at the single nucleotide level. Moreover, clinical accuracy of the SERS sensor for PC diagnosis was validated by differentiating PC patients from healthy controls using differential expression analysis of PC-associated urinary exosomal miRNAs. In particular, diagnostic performance of the biosensor of the present invention is vastly superior to that of conventional qRT-PCR, indicating the potential of the SERS-based miRNA sensor for PC diagnosis. Therefore, it is expected that the 3D SERS sensor of the present invention will serve as a versatile platform for detecting various disease-related miRNAs and other trace biomarkers such as proteins or cell-free nucleic acids in biofluids.

As is apparent from the above description, a SERS-based label-free biosensor according to the present invention is configured such that a 3D hierarchical nanoarchitecture is constructed through self-assembly of a plasmonic head-flocked gold nanopillar SERS substrate and SAP-AuNPs in the presence of target miRNA, and uniform and rich nanogaps of the 3D nanoarchitecture thus constructed lead to intense broadening of the intrinsic SERS fingerprint peak of target miRNA, resulting in ultrahigh sensitivity (about 10 aM) to target miRNA and specific detection performance at the single nucleotide level. Furthermore, clinical accuracy of the SERS sensor for PC diagnosis distinguishes PC patients from healthy controls using differential expression analysis of PC-associated urinary exosomal miRNAs. In particular, the SERS biosensor of the present invention exhibits much better diagnostic performance than conventional qRT-PCR, and thus has potential as a SERS-based miRNA sensor for PC diagnosis. It is expected that the 3D SERS sensor of the present invention will serve as a versatile platform for detecting various disease-related miRNAs and other trace biomarkers such as proteins or cell-free nucleic acids in biofluids.

From the above description, those skilled in the art to which the present invention belongs will understand that the present invention may be embodied in other specific forms without changing the technical spirit or essential characteristics thereof. In this regard, the embodiments described above should be understood to be non-limiting and illustrative in every way. The scope of the present invention should be construed as including, rather than the above detailed description, all changes or modifications derived from the meaning and scope of the following claims and equivalents thereto.

What is claimed is:

1. A surface-enhanced Raman scattering (SERS) biosensor for diagnosing prostate cancer with high sensitivity, comprising:
a SERS-based 3D hierarchical nanosubstrate with a SERS signal amplified, which comprises a head-flocked metal nanopillar structure, a capture probe specifically binding to a prostate-cancer-derived target biomarker linked to heads of the metal nanopillar structure, and metal nanoparticles with a detection probe conjugated thereto located in nanogaps between the heads of the metal nanopillar structure;
a Raman microscope to which the nanosubstrate is attached; and
a measurement unit configured to measure an amplified Raman signal with the Raman microscope.

2. The surface-enhanced Raman scattering (SERS) biosensor according to claim 1, wherein the metal nanopillar structure comprises a metal plate and metal nanopillars (heads).

3. The surface-enhanced Raman scattering (SERS) biosensor according to claim 1, wherein the metal is at least one selected from the group consisting of gold, silver, platinum, and aluminum.

4. The surface-enhanced Raman scattering (SERS) biosensor according to claim 1, wherein the head-flocked nanopillar structure generates an enhanced SERS signal by reducing a distance between nanopillar heads.

5. The surface-enhanced Raman scattering (SERS) biosensor according to claim 1, wherein the metal nanoparticles have a size of 7 to 50 nm.

6. The surface-enhanced Raman scattering (SERS) biosensor according to claim 1, wherein the nanogaps have a size of 3 to 20 nm.

7. The surface-enhanced Raman scattering (SERS) biosensor according to claim 1, wherein the biomarker is DNA, miRNA, or peptide.

8. The surface-enhanced Raman scattering (SERS) biosensor according to claim 7, wherein the miRNA is miRNA-10a or miRNA-21.

9. The surface-enhanced Raman scattering (SERS) biosensor according to claim 1, wherein the probe is 75 bp to 150 bp long.

10. The surface-enhanced Raman scattering (SERS) biosensor according to claim 1, wherein an end of each of the capture probe and the detection probe is modified with a thiol group.

11. The surface-enhanced Raman scattering (SERS) biosensor according to claim 1, wherein the probe comprises DNA or LNA.

12. The surface-enhanced Raman scattering (SERS) biosensor according to claim 1, wherein the biosensor detects an exosome-derived miRNA protein by measuring a change in Rayleigh scattering spectrum caused by specific binding of exosome-derived miRNA.

13. The surface-enhanced Raman scattering (SERS) biosensor according to claim 1, wherein the biosensor detects a prostate-cancer-derived target biomarker in a wide range of attomolar concentration (aM) to nanomolar concentration (nM).

14. A method of diagnosing prostate cancer with high sensitivity, comprising treating a biomarker mixture with the surface-enhanced Raman scattering (SERS) biosensor according to claim 1.

15. The method according to claim 14, wherein the biomarker mixture is blood or urine.

16. The method according to claim 15, wherein the biomarker mixture comprises exosome-derived miRNA.

17. A method of detecting a prostate-cancer-derived target biomarker based on surface-enhanced Raman scattering (SERS), comprising detecting a prostate-cancer-derived target biomarker using the surface-enhanced Raman scattering (SERS) biosensor according to claim 1.

18. A surface-enhanced Raman scattering (SERS) biosensor for detecting a prostate-cancer-derived target biomarker with high sensitivity, comprising:
a SERS-based 3D hierarchical nanosubstrate with a SERS signal amplified, which comprises a head-flocked metal nanopillar structure, a capture probe specifically binding to a prostate-cancer-derived target biomarker linked to heads of the metal nanopillar structure, and metal nanoparticles with a detection probe conjugated thereto located in nanogaps between the heads of the metal nanopillar structure;
a Raman microscope to which the nanosubstrate is attached; and
a measurement unit configured to measure an amplified Raman signal with the Raman microscope.
